(19) United States
(12) Patent Application Publication
(10) Pub. No.: US 2007/0036735 A1
(43) Pub. Date: Feb. 15, 2007

(54) Skin external preparation

(76) Inventor: Shinichi Kitasaki, Saitama (JP)

Correspondence Address: WENDEROTH, LIND & PONACK, L.L.P., 2033 K STREET N. W., SUITE 800, WASHINGTON, DC, 20006-1021, US

(21) Appl. No.: 10/572,456

(22) PCT Filed: Sep. 17, 2004
(86) PCT No.: PCT/JP04/14013

§ 371 Date: Jun. 14, 2006

(30) Foreign Application Priority Data
Sep. 19, 2003 (JP) 2003-328852

(51) Int. Cl. A61K 36/02

(52) U.S. Cl. 424/63; 424/195.17

(57) ABSTRACT

A skin external preparation comprises, as an effective component, the powdered of calcareous algae globes into which calcareous algae belonging to corallinaceae of red algae have grown globularly.

### CROSS-REFERENCE TO RELATED APPLICATIONS

The present application claims priority from Japanese Patent Application No. 2003-328852 filed on Sep. 19, 2003, which is herein incorporated by reference.

### TECHNICAL FIELD

The present invention relates to a skin external preparation which is made to contact a roughened skin due to skin diseases such as atopic dermatitis, eczema, and psoriasis in order to improve these skin diseases.

### BACKGROUND ART

Various kinds of substances have so far been proposed as a skin external preparation for treating atopic dermatitis. In particular, granite which radiates far-infrared rays with a wave length of 4 to 14 μm. ceramic earth (as metallic oxides, comprising about 60% of silicate, about 17% of aluminum oxide, about 12% of magnesium oxide, about 5 to 6% of ferrous oxide, and 5 to 6% of the others), tourmaline, magny, ceramics, and the like are utilized.

As disclosed in Japanese Patent Application Laid-open Publication No. H09-173479, it is also proposed to spread over the bottom of a bathtub globular products into which rocks have been pulverized which radiate far-infrared rays with a wave length of 4 to 14 μm and contain at least about 28% of a silicon, about 10% of an aluminum, and about 6% of potassium.

These substances radiating far-infrared rays are known to cut clusters of water and also suppress the production of lipid peroxide which is said to cause various diseases.

The lipid peroxide is yielded by the oxidation by active oxygen of cells or of lipid existing in interstitium between cells. Therefore, highly antioxidative substances are said to be effective for improving skin diseases such as atopic dermatitis and the like.

### DISCLOSURE OF THE INVENTION

The present invention was made in view of the technological background described above. It is an object of the invention to provide a skin external preparation which is highly antioxidative without any side-effect.

The present inventor devoted himself to seeking substances in nature which are highly antioxidative but exhibit no side-effect. As a result, he learned that the powdered of calcareous algae globes into which the calcareous algae belonging to corallinaceae of red algae have grown globularly has an extremely high antioxidative activity as compared to other substances and completed the present invention based on this learning.

Thus, the skin external preparation according to the present invention contains, as an effective component, the powdered of calcareous algae globes into which the calcareous algae belonging to corallinaceae of red algae have grown globularly.

The calcareous algae globes exist in a sand stone layer (Nanakama sand stone layer) of Oligocene Nishisonogi layer group which is distributed in the northwest area of Nishisonogi Peninsula (Saikai Town) in Nagasaki Prefecture. It has been identified that the substrate of the sand stone layer containing calcareous algae globes comprises substances such as quartz, white mica, black mica, chlorite, and plagioclase, by means of a scope observation and x-ray diffraction analysis. Grain sizes range from medium to coarse with little amount of a muddy component contained. The calcareous algae globes are mainly constructed from species of a Lithothamnion genus of Coralinaceae of red algae which are finely stacked in layers and grow to ellipse globes with a diameter of 2 to 9 cm (literature name: monthly magazine “Chikyu”, Vol. 17, No. 9, 1995 “Stacking environment/Formation age and varying factors of carbon-oxygen isotope composition of a sand stone layer containing calcareous algae globes (Oligocene Nanakama sand stone layer)”.

It is preferable to pulverize the calcareous algae globes as finely as possible, preferably into fine powder with a diameter of 5 μm or less, and mix the produced powder with an adhesive containing 50 wt% or more of petrolatum or the like so as to apply to diseased areas.

### BEST MODE FOR CARRYING OUT THE INVENTION

It has been made apparent that oxidation by active oxygen causes skin diseases such as atopic dermatitis. Therefore, in order to check an antioxidative activity of the skin external preparation according to the present invention, comparison test was first carried out on the powdered of the calcareous algae globes according to the present invention, and the powdered of magny and powdered of tourmaline of conventional art.

In this experiment, the following samples were prepared: an invented product A obtained by mixing powder of 2-3 μm into which calcareous algae globes were pulverized using a mortar with white petrolatum in a weight ratio of 3:2; and comparison products B and C, which differ from A only in that they have magny and tourmaline respectively instead of calcareous algae globes.

4.09 mg of each of these samples was applied to the inside bottom of a respective petri dish, and 5.00 g of squalene oil (C₃₀H₅₀) which was selected as a lipid susceptible to oxidation was poured from above onto each sample. The petri dish was then fitted with a lid. Then, daily variations in weight thereof were measured over time while keeping the petri dish at 37° C. in an incubator. The weights of three samples varied over time. The increase in weight was caused by the formation of lipid peroxide due to the oxidation of squalene oil, and reflected the amount of lipid peroxide. It can therefore be said that the less the increase in weight, the higher antioxidative activity the sample has. The increase in weight is shown in Table 1.

INCREASE IN LIPID PEROXIDE (mg/g)

| Sample/Days | 30 days | 37 days | 51 days |
|---|---|---|---|
| A | 148 | 173 | 195 |
| B | 148 | 180 | 210 |
| C | 185 | 210 | 228 |

The results in Table 1 shows that antioxidative power that prevents lipid peroxide from increasing is strong in the order of A, B, and C and particularly shows that the invented product A is excellent over magny and tourmaline.

Next, the clinical manifestations of atopic dermatitis and curative effects thereon were verified.

Three major symptoms of atopic dermatitis are “feeling itchy”, “being hardened”, and “being blackened”. However, the final diagnosis is decided by visual inspection carried out by a specialist. Thus, the effects of a curing ointment should be determined according to the degrees of improvement in the subjective symptoms on patients themselves and in the objective observation of the symptoms by specialists.

In this verification, the extent of the symptoms was expressed by a point value system as shown below to objectively evaluate the improvement in atopic dermatitis.
A. Itching (Jucken)
   1. Slightly itchy.
   2. Itchy but able to sleep.
   3. Sometimes awakened due to itching.
   4. Not able to sleep due to itching.
   5. Not able to imagine more terrible itching. Being so serious as to gouge the skin with their nails and not to be able to sleep at all.
B. Thickened lichen
   1. Not evident by visual inspection, but there are abnormalities recognizable by palpation.
   2. Faintly swelled and harsh.
   3. Observably swelled.
   4. Stiffly swelled and stubby.
   5. As stiff as the skin of an elephant.
C. Pigmentation
Note: Plus one point if a netted pattern is found in a neck.
   1. Dullness in the skin is found.
   2. An abnormal color is found in an area.
   3. An abnormal color is found in areas including a face.
   4. A brown color is found on the entire body.
   5. A coal color is mixed in on the entire body.
D. Nodular prurigo (P.K.)
Note: Plus one point if it is on a face.
   1. Prurigo with a half rice-grain sized convex portion is found.
   2. Prurigo with a rice-grain sized convex portion is found.
   3. Prurigo with a soybean sized hard convex portion is found.
   4. Prurigo with a broad bean sized hard convex portion is found.
   5. Prurigos as hard as a foot corn are spread in the entire body.
E. Erythema (Erythem)
Note: Plus one point if erosion is found.
   1. Limited to a frequent site of limbs.
   2. Found in a site other than the frequent site.
   3. Patient having an erythema in their face.
   4. Spread on almost the entire body.
   5. Covering the entire body without space.
F. Atopic skin
   1. Goose bumps like a broiler are found.
   2. Floury dry skin covers the entire body.
   3. Somewhat hard skin colored of a dull bronze color covers the entire body.

A calculating equation that shows the effect of treatment is shown below:

Score before starting a treatment—Score at the time of evaluation=Score indicating the effect of treatment (degree of improvement)

==Clinical Test==

The ointment according to the invention which contains the powdered of the calcareous algae globes according to the invention was applied to skins having symptoms.

(Applying Method)

This ointment was prepared by mixing the powdered of the calcareous algae globes with white petrolatum at a weight ratio of 3 to 2 to increase an adhesion degree. Selected as comparative example ones were samples which were prepared by pulverizing tourmaline and magny respectively into powder and mixing it with petrolatum just as for the ointment according to the invention, and Kindavate ointment (0.05% of clobetasone butyrate) . This Kindavate ointment is currently considered as a skin external preparation most effective to atopic dermatitis, although using it is steroid dosing (adrenal lipid hormone: IV group steroid ointment) and it has the problems of side effects including a renal lesion and an increase in a blood pressure, and of losing effects as it is used for long time period.
Patient A's case: The main symptom was nodular prurigo.

Soybean sized P.K.s (nodular prurigo) which firmly spread its hard roots and also had a height were scattered on the four limbs. Two particularly firm P.K.s were selected. The comparison was made between one P.K. painted with the ointment according to the invention and the other P.K. painted with the comparative example ointment. In this comparison, the first and second comparisons were separately made. In the first comparison, one of the right and left sides of the patient's body was painted with the ointment according to the invention and the other side was painted with Kindavate ointment. In the second comparison, one side of the right and left sides of the patient's body was painted with the ointment according to the invention and the other side of the body was painted with magny.

Also for symptom cases below, the comparison was made in the same manner as above where a plurality of comparative example ointments were used.

An external application was made once per day. Comparison was made 60 days later.

| | Before start | | At the second | | Improvement |
|---|---|---|---|---|---|
| | of test | 60 days later | diagnosis | Effect | points per day |
| Invented | 3 pts. | Thin skin was peeled. | 0 pt. | 3 pts. | 0.05 pt. |
| Ointment | | Height was reduced | | | |
| | | and hardly sensed. | | | |
| Kindavate | 3 pts. | No change | 3 pts. | 0 pt. | 0.00 pt. |
| Magny | 3 pts. | Height was reduced | 1 pt. | 2 pts. | 0.03 pt. |
| | | in half, and size | | | |
| | | was to half rice- | | | |
| | | grain size. | | | |

Patient B's case: The main symptom was erythem in an area of the neck to clavicle.

Erythem was in the face too, hence having 3 points. The extent of itching was 3 points. The total was 6 points.

Comparison was made between the right side painted with the ointment of the invention and the left side painted with the comparative example ointment (Kindavate ointment).

| | Before start | | At the second | | | Improvement |
|---|---|---|---|---|---|---|
| | of test | 4 days later | diagnosis | | Effect | points per day |
| Invented | Red, rough: | | 0 | pt. | 6 pts. | 1.5 pts. |
| Ointment | 6 pts. | | | | | |
| Kindavate | Red, rough: | Smooth, no Erythem, | 0 | pt. | 6 pts. | 1.5 pts. |
| | 6 pts. | and no itching. | | | | |
| Tourmaline | Red, rough: | Erythem was reduced | Erythem: 1.5 | pts. | 3.5 pts. | 0.88 pt. |
| | 6 pts. | in half. | Itching: 2 | pts. | | |
| | | Drying and itching | | | | |
| | | were left. | | | | |

Patient C's case: The main symptom was pigmentation in an area of the face to neck to clavicle.

An abnormal color was recognizable in an area including the face, hence having 3 points. A netted pattern was recognizable in an area of the neck to clavicle, adding 1 point. The total was 4 points.

The right and left sides with respect to the Adam's apple were painted differently.

| | Before start | | At the second | | Improvement |
|---|---|---|---|---|---|
| | of test | 30 days later | diagnosis | Effect | points per day |
| Invented | 4 pts. | No abnormal color. | 1 pt. | 3 pts. | 0.1 pt. |
| Ointment | | Slightly dulled. | | | |
| Kindavate | 4 pts. | Almost no change | 4 pts. | 0 pt. | 0.0 pt. |

Patient D's case: Thickened lichen spots spread over the entire body, and were stiffly swelled and stubby, hence having 4 points. The presence of Jucken had 3 points.

The right and left sides were painted differently and compared.

| | Before start | | At the second | | Improvement |
|---|---|---|---|---|---|
| | of test | 30 days later | diagnosis | Effect | points per day |
| Invented | 4 + 3 = 7 pts. | Softened. No itching. | 0 pt. | 3 pts. | 0.05 pt. |
| Ointment | | | | | |
| Kindavate | 4 + 3 = 7 pts. | No change. Itching | 6 pts. | 1 pt. | 0.03 pt. |
| | | was slightly reduced. | | | |
| Magny | 4 + 3 = 7 pts. | Softened. Swells | 2.5 pts. | 4.5 pts. | 0.15 pt. |
| | | were reduced in half. | | | |
| | | No itching. | | | |

Patient E's case: The main symptom was a netted pattern of pigmentation on the neck, without Jucken.

The right and left sides were painted differently and compared.

| | Symptom before | | At thesecond | | Improvement |
|---|---|---|---|---|---|
| | start of test | 14 days later | diagnosis | Effect | points per day |
| Invented | Not in the face: 2 pts. | Color disappeared, | 1 pt. | 2 pts. | 0.14 pt. |
| Ointment | Netted pattern: 1 pt. | but dullness exists. | | | |
| | Total: 3 pts. | | | | |
| Kindavate | Not in the face: 2 pts. | Almost no change | 3 pts. | 0 pt. | 0.00 pt. |
| | Netted pattern: 1 pt. | | | | |
| | Total: 3 pts. | | | | |

Patient F's case: Goose bumps like a broiler were recognizable on the limbs and body trunk. Atopic skin. The presence of Jucken had 2 points.

The right and left sides were painted differently and compared.

| | Symptom | | At the second | | Improvement |
|---|---|---|---|---|---|
| | before start | 7 days later | diagnosis | Effect | points per day |
| Invented | 2 + 2 = 4 pts. | Symptom | 0 pt. | 4 pts. | 0.57 pt. |
| Ointment | | disappeared | | | |
| Kindavate | ″ | Symptom | 0 pt. | 4 pts. | 0.57 pt. |
| | | disappeared | | | |
| Tourmaline | 2 + 2 = 4 pts. | Atopic skin | 1 + 1 = 2 pts. | 2 pts. | 0.29 pt. |
| | | and itching | | | |
| | | were partly left. | | | |

Patient G's case: A somewhat hard skin in dull bronze, etc., covered the entire body. Application of physical stimuli drew pale lines. The presence of Jucken had 2 points.

| | Symptom | | At the second | | Improvement |
|---|---|---|---|---|---|
| | before start | 14 days later | diagnosis | Effect | points per day |
| Invented | 3 + 2 = 5 pts. | Symptom disappeared | 0 pt. | 5 pts. | 0.36 pt. |
| Ointment | | | | | |
| Kindavate | ″ | Symptom disappeared | 0 pt. | 5 pts. | 0.36 pt. |
| Tourmaline | 3 + 2 = 5 pts. | Atopic skin was partly | 1 + 1 = 2 pts. | 3 pts. | 0.21 pt. |
| | | left and itching was left. | | | |

Cosmetic medicine “whitening”

So-called “fleck” and “dull spots” in the field of a cosmetic medicine are pathologically classified into a sort of pigmentation and have the same etiology as that of pigmentation. Therefore, the ointment of the invention works well to these. “Whitening” means improvement in pigmentation. A dark skin results from a sort of moderate pigmentation, and has the same etiology as that of pigmentation. Thus, the ointment of the invention works well.

Criteria for evaluating “fleck” and “dull spots”
0 points: Clean
1 point: Barely recognizable as being darker than the surrounding area.
2 points: Recognizable as being darker than the surrounding area.
3 points: Being of a biscuit color.
4 points: Being of a cafe au lait color.
5 points: Being of a coffee brown color.
Patient H's Case: Pronounced flecks and dull spots created after sunburn were scattered over the shoulder and cheek.

The right and left sides were painted differently.

| | Symptom before | | At the second | | Improvement |
|---|---|---|---|---|---|
| | start of test | 60 days later | diagnosis | Effect | points per day |
| Invented | Partly in coffee brown | Recognizable as being | 2 pts. | 2.5 pts. | 0.04 pt. |
| Ointment | and of cafe au lait | darker than the | | | |
| | color: 4.5 pts | surrounding area | | | |
| Kindavate | Partly in coffee brown | No change | 4.5 pts. | 0.0 pt. | 0.00 pt. |
| | and of cafe au lait | | | | |
| | color: 4.5 pts | | | | |

Patient I's case: Flecks were clearly recognizable in the face. Numerous flecks were recognizable through careful observation. The right and left sides with respect to the face center line were painted differently.

| | Symptom before | | At the second | | Improvement |
|---|---|---|---|---|---|
| | start of test | 30 days later | diagnosis | Effect | points per day |
| Invented | 3 pts. | Flecks were | 1 pt. | 2 pts. | 0.07 pt. |
| Ointment | | reduced | | | |
| Kindavate | 3 pts. | No change | 3 pts. | 0 pt. | 0.00 pt. |

Patient J's case: Senile flecks were clearly recognizable on the cheek.

Since they were on both right and left sides, the right and left sides were painted differently.

| | Symptom before | | At the second | | Improvement |
|---|---|---|---|---|---|
| | start of test | 30 days later | diagnosis | Effect | points per day |
| Invented | 4 pts. | Flecks were | 2 pts. | 2 pts. | 0.07 pt. |
| Ointment | | greatly reduced | | | |
| Kindavate | 4 pts. | No change | 4 pts. | 0 pt. | 0.00 pt. |

Criteria for evaluating “whitening”

| | 0 | 1 | 2 | 3 |
|---|---|---|---|---|
| Skin cond. from | White | Without | Skin pores | Dry scaly |
| viewpoint of | skin | a problem | stand out. | skin. With |
| cosmetic medi- | | | Skin texture | a Honeycomb |
| cine | | | is coarse. | pattern. Dry |
| | | | | skin |

Patient K's case: Dry skin areas having a honeycomb pattern stood out on four limbs, particularly on the outside of the legs. Skin texture was coarse on the entire body. The right and left sides of the entire body were painted differently.

| | Symptom | | | | |
|---|---|---|---|---|---|
| | before | | At the | | Improve- |
| | start | | second | | ment points |
| | of test | 60 days later | diagnosis | Effect | per day |
| Invented | 3 pts. | Skin was | 1 pts. | 2 pts. | 0.07 pt. |
| Ointment | | moisturized | | | |
| Kindavate | 3 pts. | No change | 3 pts. | 0 pt. | 0.00 pt. |

Patient L's case: The patient said, “The face has darkened after overseas travel, and the color would not go away.” The color appeared normal without a problem objectively. The right and left sides with respect to the face center line were painted differently. The second diagnosis one month later showed that the side painted with the ointment of the invention was clearly whiter.

| | Symptom | | | | |
|---|---|---|---|---|---|
| | before | | At the | | Improve- |
| | start | | second | | ment points |
| | of test | 60 days later | diagnosis | Effect | per day |
| Invented | 1 pt. | Whitened | 0 pt. | 1 pt. | 0.03 pt. |
| Ointment | | | | | |
| Kindavate | 1 pt. | No change | 1 pt. | 0 pt. | 0.00 pt. |

As apparent from the above cases showing treatment effects, the use of the ointment according to the present invention exhibits treatment effects greater than or equivalent to Kindavate ointment which is a steroidal ointment. It can be said that the present invention uses the powdered of calcareous algae globes which are a natural substance, thus not having side-effects like those of steroid but exhibiting extremely great effects.

### INDUSTRIAL APPLICABILITY

The skin external preparation of the invention improves skin diseases such as atopic dermatitis, eczema, psoriasis, and the like without any side-effect, and also can produce the whitening effect.

1. A skin external preparation comprising, as an effective component, the powder of calcareous algae globes into which calcareous algae belonging to corallinaceae of red algae have grown globularly.

2. The skin external preparation according to claim 1, wherein a substance separated from a sand stone layer (Nanakama sand stone layer) of Oligocene Nishisonogi layer group distributed in the northwest area (Saikai Town) of Nishisonogi Peninsula in Nagasaki Prefecture of Japan is used as the calcareous algae globes.

3. The skin external preparation according to claim 1, comprising 50% or more by weight of the powder of the calcareous algae globes.

4. The skin external preparation according to claim 3, wherein petrolatuma is added as an adhering agent to the calcareous algae globes and mixed therewith.

5. The skin external preparation according to claim 1, wherein the powder of the calcareous algae globes is a powder of 5 μm or less in average grain diameter.

6. The skin external preparation according to claim 2, comprising 50% or more by weight of the powder of the calcareous algae globes.

7. The skin external preparation according to claim 2, wherein the powder of the calcareous algae globes is a powder of 5 μm or less in average grain diameter.

8. The skin external preparation according to claim 3, wherein the powder of the calcareous algae globes is a powder of 5 μm or less in average grain diameter.

9. The skin external preparation according to claim 4, wherein the powder of the calcareous algae globes is a powder of 5 μm or less in average grain diameter.

10. The skin external preparation according to claim 6, wherein the calcareous algae globes is a powder of 5 μm or less in average grain diameter.
